(19) United States
(12) Patent Application Publication
(10) Pub. No.: US 2023/0157685 A1
(43) Pub. Date: May 25, 2023

(54) DEVICE AND ASSEMBLY FOR REPAIRING SOFT TISSUES, FOR EXAMPLE TENDONS AND LIGAMENTS

(71) Applicants: POLITECNICO DI TORINO, Torino (IT); UNIVERSITA' DEGLI STUDI DI TRENTO, Trento (IT)

(72) Inventors: Cecilia SURACE, Torino (IT); Marco CIVERA, Chieri (Torino) (IT); Mariana RODRIGUEZ REINOSO, Torino (IT); Oliver GRIMALDO RUIZ, Torino (IT); Emilio PAOLUCCI, Torino (IT); Federica BERGAMIN, Torino (IT); Nicola Maria PUGNO, Trento (IT)

(21) Appl. No.: 17/916,792

(22) PCT Filed: Apr. 1, 2021
(86) PCT No.: PCT/IB2021/052721

§ 371 Date: Oct. 3, 2022

(30) Foreign Application Priority Data
Apr. 2, 2020 (IT) 102020000006967

(51) Int. Cl. A61B 17/064

(52) U.S. Cl. CPC A61B 17/0642; A61B 17/0643; A61B 2017/00004

(57) ABSTRACT

A bio-compatible and bio-resorbable implantable device for repairing soft tissue which has a first plate and a second plate and a plurality of connecting elements for connecting the first and second plates is provided. The first plate has a first surface suitable for being placed on a first side of the soft tissue. The second plate has a second surface suitable for being placed on a second side of the soft tissue. Each connecting element has a first portion integral with the first plate and a second portion integral with the second plate. The connecting elements extend from at least one of the first surface of the first plate and the second surface of the second plate to reach the other one of the first surface of the first plate and second surface of the second plate for locking the first and second plates in a definable respective position.

### SUMMARY OF INVENTION

The subject matter of this invention is a bio-compatible and bio-resorbable implantable device for repairing soft tissues.

In particular, the implantable device is suitable for repairing soft tissues subjected to tensile loads under physiological conditions. The implantable device is particularly suitable, although not uniquely intended, for tendon repair. The device may be used in the field of ligament injuries.

This invention also relates to an assembly comprising said bio-compatible and bio-resorbable implantable device and a non-implantable element for perforating the soft tissue.

### PRIOR ART

Tendons are made up of very strong, poorly elastic fibrous tissue, the function of which is to transmit the contractile action exerted by a muscle to the corresponding skeletal segment. Other types of soft tissue subjected to tensile loads are formed of ligaments.

Fibrous tendon tissue is mainly made up of type I collagen chains helically wound to form a set of fibers aligned in the transmission direction of the load.

Tendons may be injured in various ways: direct trauma (cuts, crushing, lacerations, etc.) or indirect trauma (violent muscle contractions, sudden flexion or counter-resistance of a joint, etc.); athletes and dancers in particular are exposed to indirect trauma and the tendons most frequently involved are the Achilles tendon, the patellar-femoral tendon, the biceps, and the flexors of the fingers. Tendons may also deteriorate as a result of excessive fatigue, as may occur in the case of dancers, in degenerative diseases or the like, and/or related causes. Some additional areas of particular interest are: the hand flexors, foot flexors, tibialis anterior, patellar tendon, and the tendons of the quadriceps, biceps, and rotator cuff.

In most cases, the tendon must be surgically repaired to heal; the procedure involves making an incision in the skin, isolating and bringing the tendon stumps together and keeping them in contact until the tendon is completely healed; healing occurs through the formation of scar tissue, which is less resistant, and “neo-tendon” tissue, i.e., that which is functionally and histologically similar to the healthy tendon. The relationship between the two regenerated tissues is directly linked to the type of surgical repair and to the rehabilitation methods.

Surgical suturing (thread and needle) with resorbable or non-resorbable material is currently the most used system to repair tendon injuries; it has a low cost, is easily available, does not require dedicated tools for its application, and currently has a more favorable cost-benefit index than the other treatments on the healthcare market. However, surgical suturing has a number of drawbacks: it promotes the formation of scar tissue both inside and outside the tendon resulting in lower resistance to tensile forces of the repaired tissue and increased resistance to the sliding of the tendon and to the development of the joint movement; the locking point of the thread (knot) is the main area of weakness to tensile forces of the system with re-ruptures of the tendon possible; both the thread and the knot determine, at the point of repair, an increase in the size of the tendon in a measure directly proportional to the increase in the caliber of the thread required, with potential repercussions on the tissues adjacent thereto, without thereby leading to an improvement in the mechanical properties of the tendon.

The various suturing techniques may require a super-specialized preparation because they are not easy to perform. The suture does not ensure a sufficient grip for active joint mobilization and, depending on the tendons, passive mobilization for three or four weeks, with a resulting delay in physiotherapy and increased complications such as pain, joint stiffness, failure to recover movement, need for follow up surgery. For these reasons, patients who suffer a tendon lesion are forced into a long period of disability, require prolonged physiotherapy, and in no small percentage of cases do not fully recover the functionality of the affected area.

Naturally, even in the case of animals, especially competition animals, such as for example horses, the aforementioned problems are encountered mutatis mutandis.

The need is therefore strongly felt to provide an improved solution with respect to conventional surgical sutures for the treatment of traumatic injuries of the tendons.

In place of traditional surgical sutures, systems have been proposed that use staples, rivets, pins, or other metal retaining elements, which obviously do not solve the problems described above with reference to sutures, especially with regard to the invasiveness of the inserted device, the potential to cause inflammation and infection, and a non-optimal distribution of stresses, being in the best of cases only simpler for an operator, for example a surgeon, to apply.

For example, U.S. Patent Application No. US-2015-0173737 discloses a solution for repairing an injured tendon consisting of an elongated element intended to be inserted inside both stumps of the tendon to be repaired to act substantially as a retaining element between the two stumps. Sutures or other fastening systems of the elongated element allow it to be fastened to the tendon stumps, which otherwise would tend to move away in the event of muscle contractions in that area. This solution is therefore very invasive and requires making a longitudinal cavity to insert the elongated element inside the body of the two tendon stumps to be brought together, which then must be sutured or riveted to the tendon, causing further permanent inflammation. The application procedure is also complex and entails a risk of injury for the surgeon.

For example, U.S. Patent Application No. US-2003-0065360 shows a bandage intended to be wrapped around a tendon and provided with sharp clamping barbs designed to be individually anchored to the tissue to be repaired to secure the bandage to the tissue, substantially acting as a clamping band. This solution, if applied to tendon repairs, would not be without drawbacks. For example, the tips of the clamping barbs of the bandage would act as needles resulting in a source of further localized, as well as distributed, inflammation, due to the large number of such clamping barbs, in the tissue of the tendon to be repaired. Or worse, in the event that such needles or barbs infiltrate between the longitudinal fibers of the tendon to be repaired, the clamping could even fail. The sharp barbs are further arranged in parallel rows, i.e., along the same tendon fiber, and, during muscle contraction and tensioning of the cuffed tendon, this would likely cause mechanical stress focused on a small number of fibers, easily causing the fraying and/or the longitudinal separation of the tendon fibers, as well as the removal of said sharp barbs.

For example, U.S. Patent Application No. US-2018-0168798 in the name of CABLE FIX shows a solution formed by a pair of rigid metal plates linked together with a wire or cable that tends to urge the two plates towards each other through the body of the tissue to be repaired. Spacer elements extend through the body of the tissue latching the two plates together and are provided with relevant sliding grooves for the plates having abutment surfaces which limit the approach between the two plates by keeping them at a certain distance. Sharp perforating needles are provided on one of the two plates to penetrate the tendon tissue. This solution does not solve the aforementioned problems relating to sutures due to the provision of said prestressed cables. The prestress exerted by these cables constantly presses to compression the cross section of the tissue between the plates. In addition, the sharp needles are a source of further inflammation and damage to the tissue to be repaired.

For example, Italian patent application No. IT-2018-000006092 in the name of the same Applicant shows a clamping band solution to be wrapped around a tendon provided with barbs for anchoring to the tendon stumps to be repaired. This solution, although advantageous from some points of view and in particular due to its intrinsic compatibility with biological tissues, is not suitable for satisfactorily stimulating the regeneration of the native fibrous tissue of the tendon, imposing long healing times, which are often incompatible with the contingent professional needs of athletes and dancers.

The need is therefore strongly felt to provide a solution to repair a soft tissue, for example a tendon or a ligament, which is of reduced invasiveness with respect to known solutions, easy to apply through surgery, and at the same time suitable for allowing a rapid and complete recovery of the functionality of the tissue while also ensuring the mechanical resistance necessary for the entire duration of the rehabilitation phase.

The need is also felt to provide a solution to repair damaged soft tissue, for example a tendon or a ligament, in a shorter time, without being more invasive or even worse, resulting in an unsatisfactory functional recovery of the tissue.

The need is felt for a solution which is definitive and therefore avoids a second surgical intervention for its extraction and which does not damage the soft tissue over long periods if it remains in place.

### SOLUTION

One object of this invention is to remedy the drawbacks of the prior art heretofore attested with reference to the state of the art.

A particular object of this invention is to devise a bio-compatible and bio-resorbable device, minimally invasive and suitable for allowing a rapid and complete healing of the soft tissue, such as for example a damaged tendon.

This and other objects are achieved with a device according to claim 1, as well as with an assembly according to claim 11.

Some advantageous embodiments are the subject of the dependent claims.

According to an aspect of the invention, a bio-compatible and bio-resorbable implantable device for repairing a soft tissue, for example a tendon, comprises at least two plates that may be interlocked by means of a plurality of connecting elements which act as elements for positioning the plates with respect to each other and with respect to the tissue to be repaired. The at least two plates and the connecting elements are all bio-compatible and bio-resorbable. The connecting elements extend from one plate to the other plate with the purpose of locking said first plate and said second plate in a definable respective position, avoiding the need for suture threads.

According to an aspect of the invention, an assembly comprises at least one bio-compatible and bio-resorbable implantable device and a non-implantable perforating device made as a separate piece from said bio-compatible and bio-resorbable implantable device, wherein said perforating device comprises a plurality of perforating elements suitable for making perforations, preferably through perforations, in said soft tissue to form positioning paths for said plurality of connecting elements of the bio-compatible and bio-resorbable implantable device. The perforating device may be made in the form of another non-implantable plate.

The implantable device may also provide a possible functional coating with growth factors or with drugs that promote and speed up the process of forming autologous tissue, thus decreasing the risk of excessive scar tissue formation. These substances do not necessarily have to be in the form of a coating, but rather may be contained or encapsulated within nanoparticles, which may be inserted or incorporated within the same material and therefore distributed over the entire volume of the implantable device.

The connecting elements comprise a first portion integral with the first plate and a second portion integral with the second plate. For example, a first connecting element portion is formed by a pin and the second connecting element portion is formed by a rim of a hole.

The connecting elements may be of equal shape and size to each other, as well as made of the same material composition.

The interlocking of two portions of a connecting element and in this way of the plates to each other may take place by means of undercut coupling.

The tips of the pins or protrusions of the connecting elements may be rounded to avoid injuring the tissue to be repaired. The lateral surface of the connecting elements may be rounded.

The perforating elements may each define a longitudinal through channel to allow the insertion of the connecting elements of the bio-compatible and bio-resorbable implantable device, whereby the perforating elements are fitted on the first portion or on the second portion of the connecting elements during implantation of said first plate or of said second plate, respectively.

The connecting elements may each define a longitudinal through channel for inserting the perforating elements of the perforating device, whereby the connecting elements are fitted onto the perforating elements during the implantation of at least one of said first plate or said second plate.

The perforating elements may be made in separate pieces with respect to the support of the perforating device and fastened thereto, for example by means of threaded fastening means.

By virtue of the proposed solutions, a bio-compatible and bio-resorbable implantable device is provided, suitable to act as an element for transmitting the tensile load to injured soft tissue, for example a tendon, thus avoiding tensile stress on the length of soft tissue in the healing phase.

By virtue of the proposed solutions, it is possible to uniformly distribute the tensile load in the cross section of the soft tissue, for example a tendon, during the autologous tissue regeneration or self-repair phase.

The bio-compatibility of the material of the implantable device allows for unfavorable interactions between said material of the implantable device and the surrounding tissues to be avoided, ensuring the formation of autologous tissue and self-repair, without inducing the excessive formation of scar tissue and inflammatory processes related thereto.

By virtue of the proposed solutions, a non-implantable perforating device is provided, suitable to guide the minimally invasive insertion of the bio-compatible and bio-resorbable implantable device into the soft tissue to be repaired, for example a tendon. By virtue of the proposed solutions, a bio-compatible and bio-resorbable implantable device is provided, suitable to fully degrade within the organism of a human or animal patient within a reasonable time for the tissue repair of the soft tissue to be repaired, for example a tendon, avoiding the release of solid residues at the implantation site. The provision of such a bio-compatible and bio-resorbable implantable device avoids the need to extract the implantable device from the implantation site.

By virtue of the proposed solutions, the patient is placed in the conditions to achieve a faster and more satisfactory functional recovery.

By virtue of the proposed solutions, the risk of excess scar tissue forming in the soft tissue to be repaired is avoided.

By virtue of the proposed solutions, a physiological imbibition of the soft tissue to be repaired is allowed at or near the tissue lesion, promoting in an unusual way the regeneration of the soft tissue, for example tendon regeneration.

The bio-compatible and bio-resorbable implantable device is particularly suitable, although not uniquely intended, for implantation in a human patient, for example to repair the Achilles tendon as well as the patellar-femoral tendon, biceps, finger flexors, hand flexors, foot flexors, anterior tibialis, patellar tendon, and tendons of the quadriceps, biceps, and rotator cuff.

The bio-compatible and bio-resorbable implantable device is also suitable for implantation in an animal patient, such as a racehorse.

### FIGURES

Further features and advantages of the implantable device and of the assembly according to the invention will appear from the description given below of its preferred embodiments, given by way of non-limiting example, with reference to the attached figures, wherein:

FIG. 1 is a perspective view showing a portion of a soft tissue to be repaired and a bio-compatible and bio-resorbable implantable device inserted in said soft tissue, according to an embodiment;

FIG. 2 is a schematic perspective view of a bio-compatible and bio-resorbable implantable device, according to an embodiment, implanted to repair a tendon, shown by way of example as completely severed into two stumps;

FIG. 3 is a perspective view showing in separate parts a bio-compatible and bio-resorbable implantable device, according to an embodiment;

FIG. 4 is a sectional view taken according to the cutting plane indicated by the arrows IV of FIG. 3;

FIG. 5A shows a perspective view of a perforating device, according to an embodiment;

FIG. 5B is a sectional perspective view made according to the cutting plane indicated by the arrows V of FIG. 5A;

FIG. 5C is a plan view according to the point of view indicated by the arrow C of FIG. 5B;

FIG. 6A-6C show in schematic section the implantation of a bio-compatible and bio-resorbable implantable device shown in FIG. 3 (with transparent soft tissue for clarity);

FIG. 7 is a perspective view showing in separate parts a bio-compatible and bio-resorbable implantable device, according to an embodiment;

FIG. 8 is a sectional view taken according to the cutting plane indicated by the arrows VIII of FIG. 7;

FIG. 9A-9D show in schematic section the implantation of a bio-compatible and bio-resorbable implantable device shown in FIG. 7 (with transparent soft tissue for clarity);

FIG. 10 is a perspective view showing in separate parts a bio-compatible and bio-resorbable implantable device, according to an embodiment;

FIG. 11 is a sectional view taken according to the cutting plane indicated with the arrows XI of FIG. 10;

FIG. 12A-12D show in schematic section the implantation of a bio-compatible and bio-resorbable implantable device shown in FIG. 10 (with transparent soft tissue for clarity);

FIG. 13A shows a perspective view in separate parts of a perforating device, according to an embodiment;

FIG. 13B shows in section a portion of the perforating device shown in FIG. 13A;

FIG. 14A shows a perspective view in separate parts of a perforating device, according to an embodiment;

FIG. 14B shows in section a portion of the perforating device shown in FIG. 14A.

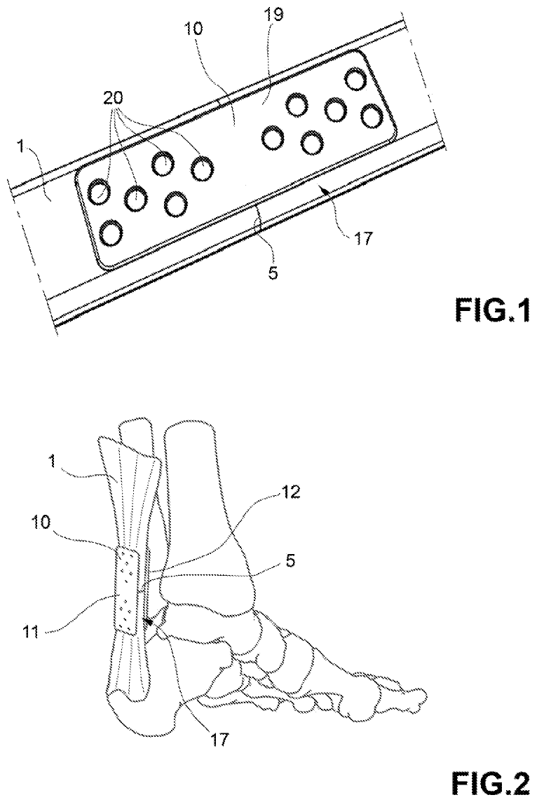

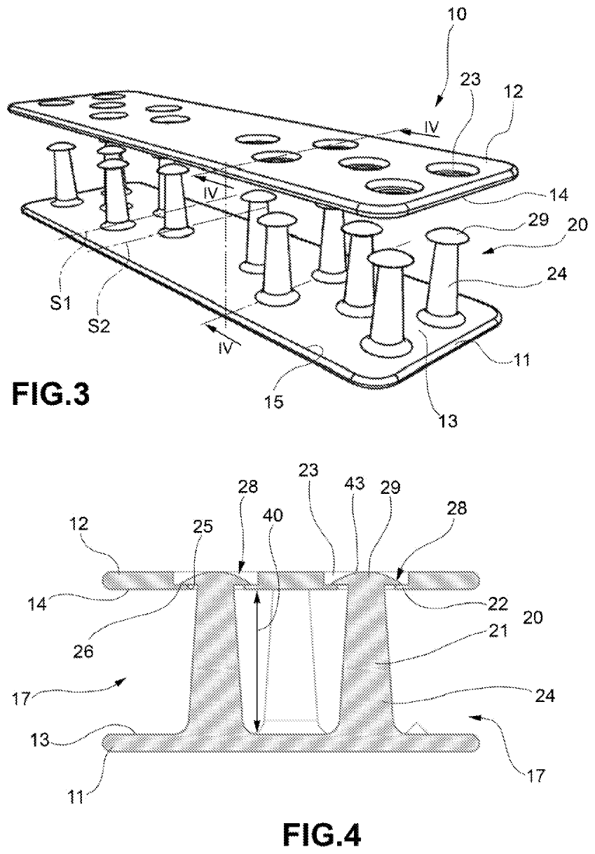

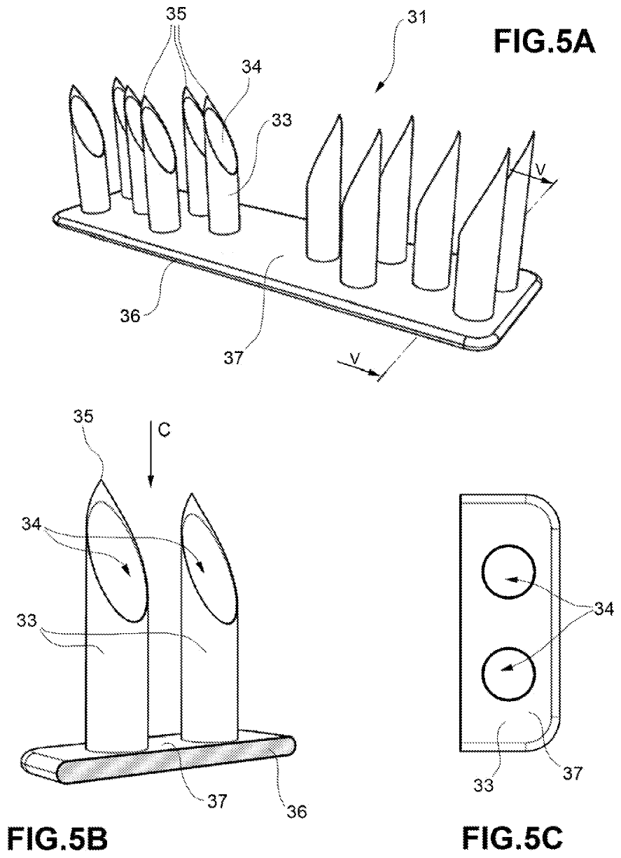

### DETAILED DESCRIPTION OF SOME EMBODIMENTS

According to a general embodiment, a bio-compatible and bio-resorbable implantable device 10 is provided for repairing a soft tissue 1, and preferably for repairing a tendon 1.

“Soft tissue 1” preferably refers to a soft tissue suitable to be stressed in extension when in physiological conditions, such as for example a tendon 1.

In the following description the term “tendon 1” will be used, referring, where applicable, also to “ligament 1.” The bio-compatible and bio-resorbable implantable device 10 is also suitable for repairing ligament tissue 1.

The implantable device 10 comprises at least two bio-compatible and bio-resorbable plates 11, 12 comprising a first plate 11 and a second plate 12.

The plates 11, 12 are made in separate pieces from one another and are interlockable with each other.

The first plate 11 may be made in a single piece. The second plate 12 may be made in a single piece.

The implantable device 10 further comprises a plurality of bio-compatible and bio-resorbable connecting elements 20 suitable for connecting said first plate 11 and said second plate 12 together. For example, said bio-compatible and bio-resorbable connecting elements 20 comprise pins 24, 24′, 24″ and hole rims 23 which receive a portion of said pins 24, 24′, 24″.

The tendon 1 to be repaired comprises a tendon body 2 which is pathologically interrupted forming a tendon lesion 5. The tendon lesion 5 may involve the entire cross section of the tendon 1 defining two tendon stumps or only a part thereof.

The tendon lesion 5 may refer to a stretch of tendon 1 not necessarily interrupted and therefore not having clear geometric discontinuity but nevertheless unable to effectively transmit the forces in the length concerned.

The first plate 11 comprises a first surface 13 suitable for being placed on a first side 3 of the tendon 1 to be repaired. Preferably, the first surface 13 of the first plate 11 is placed on a portion of the first tendon side 3 which comprises said tendon lesion 5. In other words, the first surface 13 is placed on the tendon lesion 5, substantially straddling said lesion.

The second plate 12 comprises a second surface 14, suitable for being placed on a second side 4 of the tendon 1 to be repaired, opposite to said first side 3 of the tendon 1 to be repaired with respect to the body 2 of said tendon to be repaired. Preferably, the second surface 14 of the second plate 12 is placed on a portion of the second tendon side 4 which comprises said tendon lesion 5. In other words, the second surface 14 is placed on the tendon lesion 5, substantially straddling said lesion.

In this way, the first plate 11 and the second plate 12 of the implantable device 10 are intended to be implanted whereby they are mutually contraposed and facing each other and separated by the body 2 of the tendon 1 to be repaired in the portion where the tendon lesion 5 is present. In other words, the first plate 11 and the second plate 12 are both arranged to cover the tendon lesion 5 on opposite sides 3, 4 of the body 2 of the tendon 1. Preferably, the body 2 of the tendon 1 is formed of a plurality of fibers or filaments which extend in the preferential direction of load transfer of said tendon 1, and the lesion 5 may be extended in a direction transverse to the preferential direction of load transfer, i.e., transversely to the fibers. The lesion 5 may be extended also at least partially substantially aligned with the fibers of the soft tissue 1 to be repaired, such as for example a tendon 1.

Advantageously, said plurality of connecting elements 20 of the implantable device 10 extend from at least one of said first surface 13 of the first plate 11 or said second surface 14 of the second plate 12 to reach the other of said first surface 13 of the first plate 11 and said second surface 14 of the second plate 12.

Preferably, the length of these connecting elements 20 is so as not to protrude beyond the back of the plates 11, 12, avoiding causing friction with the surrounding tissues which could inflame these surrounding tissues and could hinder the movement of the implantable device 10 integral with the tendon 1 during physiological movements of the tendon 1.

With a further advantage, the purpose of said plurality of connecting elements 20 is to lock said first plate 11 and said second plate 12 in a definable respective position.

In this way, the positioning elements 20 act as positioning elements for the plates 11, 12 with respect to the tendon 1 to be repaired.

Preferably, the plates 11, 12 are locked together in a respective configuration by the connecting elements 20, avoiding the presence of residual degrees of freedom of mutual movement between the plates 11, 12.

The plates 11, 12 therefore perform the dual function of transmitting forces in the tract wherein they are not transmissible along the injured tendon, as well as maintaining the relative position of the tendon tracts, maintaining the diastasis between the two tendon stumps within a physiological distance which allows tissue regeneration between said two stumps.

The first surface 13 and the second surface 14 intended to come into contact on opposite sides 3, 4 of the tendon 1 to be repaired may preferably be worked so as to be made smooth, thus reducing the risk of inflammation of the tendon to be repaired due to chafing by friction.

According to an embodiment, at least some connecting elements of said plurality of connecting elements 20 of the implantable device 10 extend in the form of protrusions 24, 24′, 24″ from the first surface 13 of the first plate 11.

According to an embodiment, at least some connecting elements of said plurality of connecting elements 20 of the implantable device 10 extend in the form of protrusions 24, 24′, 24″ from the second surface 14 of the second plate 12.

Preferably, when the implantable device 10 is implanted, each connecting element 20 extends from said first surface 13 to said second surface 14, although at least one connecting element or each connecting element 20 may be made in at least two separate pieces, i.e. a first piece 21 or first portion 21 integral with the first plate 11 and a second piece 22 or second portion 22 integral with the second plate 12, defining a locking portion 28 which, when the implantable device 10 is implanted, may be embedded in the body 2 of the tendon 1.

In the event that at least one connecting element or each connecting element extends from said first surface 13 to said second surface 14 without interruption, the locking portion 28 will be placed near one of said first plate 11 or said second plate 12. In this case, for example, the second portion 22 of the connecting element 20 will be formed by the rim of a hole 23 integral with the second plate 12.

According to a preferred embodiment, each connecting element of said plurality of connecting elements 20 comprises two portions 21, 22 which are interlockable with each other, forming a plurality of locking portions 28. In this way, it is possible to lock said first plate 11 and said second plate 12 together.

The portions of each connecting element 20 may interlock in various ways.

According to an embodiment, the two portions 21, 22 of a connecting element 20 interlock by undercut coupling, wherein a first portion 21 of the connecting element integral with the first plate 11 comprises a first abutment surface 25 facing the first surface 13 of the first plate 11, so as to couple against a second abutment surface 26 of the second plate 12 opposed or contraposed to the second surface 14 of the second plate 12. For example, the second abutment surface 26 may be placed on the back 19 of the second plate 12.

According to an embodiment, said protrusions 24, 24′, 24″, which form at least one of said first portion 21 or said second portion 22 of each connecting element 20, comprise a head 29 having a tip 43 which is convex. The provision of the convex tip 43 of the head 29 of a first or second portion 21, 22 of a connecting element avoids inflaming the tendon 1 to be repaired during the implantation of the bio-compatible and bio-resorbable implantable device 10.

According to an embodiment, said protrusions 24, 24′, 24″, which form at least one of said first portion 21 or said second portion 22 of each connecting element 20, a convex base 42, for example substantially circular, and a lateral surface 44 without sharp edges, for example cylindrical or frusto-conical. The provision of a first or second portion 21, 22 of a connecting element without sharp edges, for example substantially frusto-conical or cylindrical, avoids inflaming the tendon 1 to be repaired.

The lateral surface may extend from the base 42 to the head 29. The head 29 may form an undercut surface facing the base 42 which acts as the abutment surface 25 or 26. For example, the lateral surface and the head 29 form a substantially mushroom-shaped element.

As shown for example in FIG. 4, the first portion 21 of the connecting element comprises a head 29, preferably having a circular base 42 and a convex tip 43 and forming a first abutment surface 25 facing the first surface 13 of the first plate 11, the first abutment surface 25 engages in an undercut against a second abutment surface 26 of the second plate 12 facing opposite to the second surface 14 of the second plate 12. In this case, the second portion 22 of the connecting element integral with the second plate 12 is formed of the rim of the hole 23.

According to an embodiment, the two portions of a connecting element 20 interlock by latching.

According to an embodiment, the two portions of a connecting element 20 interlock by snap-fitting following an elastic deformation of a portion of the connecting element.

According to an embodiment, the two portions of a connecting element 20 interlock by force fitting or interference fitting, wherein the deformation of at least a portion of the connecting element interlocks with the first plate 11 and the second plate 12 by friction.

According to an embodiment, the two portions of a connecting element 20 interlock by hook-loop coupling, such as, for example, by means of Velcro®.

According to an embodiment, the two portions of a connecting element 20 interlock by a combination of the methods described above.

When the implantable device 10 has been implanted and has been bio-resorbed, it will be dissolved, and the body 2 of the tendon 1 will have healed the lesion 5 and is preferably indistinguishable from said tendon before the formation of the lesion 5.

According to an embodiment, said first plate 11 and said second plate 12 each have a closed and continuous plate edge 15, 16, wherein the edge 15 of the first plate 11 defines the perimeter of said first surface 13 of the first plate 11, and wherein the edge 16 defines the perimeter of said second surface 14 of the second plate 12.

According to an embodiment, said plurality of connecting elements 20 extend from said first surface 13 of the first plate 11 to said second surface 14 of the second plate 12 substantially straight, i.e., in a substantially straight line, in a direction transverse to the extension of the plates 11, 12. The substantially straight extension lines of the connecting elements are preferably parallel to each other, minimizing the length of these connecting elements.

In this way, it is possible to maximize the resistance of the bio-compatible and bio-resorbable connecting elements 20 and likewise the resistance that the implantable device 10 offers to the respective distancing of the margins of the lesion 5 of the tendon 1 to be repaired.

According to an embodiment, at least some connecting elements of said plurality of connecting elements 20 are formed by a blind or through hole 23 positioned on said first plate 11 or said second plate 12 and by a protrusion 24 positioned on the other of said first plate 11 or said second plate 12 in a position facing said blind or through hole 23.

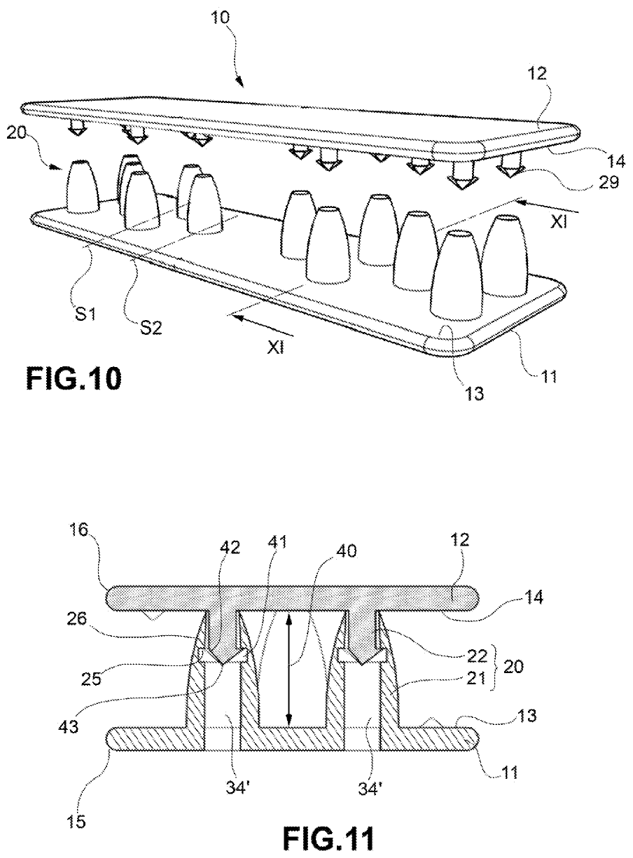

According to an embodiment, at least some connecting elements of said plurality of connecting elements 20 are formed of a first protrusion 24′, which extends from said first plate 11, and a second protrusion 24″, which extends from said second plate 12 in a position facing said first protrusion 24′, wherein said first protrusion 24′ defines said blind or through hole 23, and wherein said second protrusion 24″ defines said head 29, which, as shown for example in FIG. 10, may have a polygonal base 42 and a sharp tip portion 43. Preferably, said first protrusion 24′ defines said hole 23 in a substantially discoidal internal seat cavity 41, forming at least one first abutment surface 25 contraposed with respect to the first surface 13 of the first plate 11. Preferably, said second protrusion 24″ defines a second abutment surface 26 facing said second surface 14 of the second plate 12.

According to an embodiment, said plurality of connecting elements 20 are arranged on arrays S1, S2 or rows, wherein the connecting elements of a first array S1 are arranged staggered with respect to the connecting elements of a second array S2 contiguous to said first array S1. Preferably, the arrays S1, S2 follow each other in a direction transverse to the direction of extension of the plurality of connecting elements 20 and transverse to the preferential direction of transmission of the forces of said tendon 1.

The lateral surfaces 44 of the connecting elements 20 form positioning abutments for the fibers of the tendon 1 to be repaired which promote the healing of the lesion 5. The lateral surfaces of the connecting elements 20 are preferably curved, avoiding sharp edges which could damage the body 2 of the tendon to be repaired both during implantation and when implanted inside the body 2 of the tendon 1 to be repaired.

The curved lateral surfaces of the connecting elements 20 also allow the mechanical strength of the connecting elements to be maximized.

According to an embodiment, said plurality of connecting elements 20 keep said first surface 13 of the first plate 11 and said second contraposed surface 14 of said second plate 12 apart by a distance 40, forming one or more windows 17 delimited at least partially by both said first plate 11 and said second plate 12 and suitable for exposing a portion of the tendon 1 to the surrounding environment. Preferably, said one or more windows 17 are delimited by both plate edges 15, 16 of said first and second plate 11, 12.

The windows 17 allow for the vascularization of the tendon 1 at or near the lesion 5 when the implantable device 10 has been implanted.

According to a general embodiment, an assembly 30 for repairing a soft tissue 1, for example a tendon 1, comprises at least one bio-compatible and bio-resorbable implantable device 10 according to any of the embodiments described above.

Said assembly 30 further comprises a non-implantable perforating device 31 made in a separate piece with respect to said bio-compatible and bio-resorbable implantable device 10. Preferably, said perforating device 31 is made in the form of a third perforating plate 31.

Said perforating device 31 comprises a plurality of perforating elements 33 suitable for making through perforations 32 in said tendon 1 to form positioning paths for said plurality of connecting elements 20 of the bio-compatible and bio-resorbable implantable device 10.

According to an embodiment, said plurality of perforating elements 33 each delimit a longitudinal through cavity 34 for the insertion of at least one portion 21 or 22 of the connecting elements of said plurality of connecting elements 20 of the bio-compatible and bio-resorbable implantable device 10. In this way, the perforating elements are fitted onto the connecting elements 20 during implantation of the implantable device 10.

According to an embodiment, said at least one portion 21 or 22 of the connecting elements 20 of said plurality of connecting elements 20 delimits a longitudinal through cavity 34′ for the insertion of said plurality of perforating elements 33 of the perforating device 31. In this way, the connecting elements 20 of the implantable device 10 are fitted onto the perforating elements 33 of the perforating device 31, during the implantation of the implantable device 10.

According to an embodiment, said perforating device 31 comprises a support 36 from which said perforating elements 33 extend. The support 36 is preferably a plate so that the perforating device 31 forms a further third plate 31 of the assembly 30.

Preferably, the perforating elements 33 each comprise a sharp perforating end 35, obtained for example on the cylindrical rim of each perforating element 33.

According to an embodiment, the perforating elements 33 are made in separate pieces with respect to the support 36 and fixed thereto through fastening means. Said means for fastening the perforating elements 33 to the support 36 are preferably threaded fastening means of the screw-nut type, whereby the perforating elements 33 are screwed to the support 36.

According to an embodiment, each perforating element 33 is individually screwed to the support 36 by means of a fastening screw 38. The term “screw” also refers to a fastening grub screw 38. According to an embodiment, each perforating element 33 comprises a fastening root 45 opposite to the sharp perforating end 35 screwed to the support 36. The fastening root 45 may be screwed to the support 36 by tapping the fastening root 45. Preferably, the fastening root 45 is screwed to the support 36 by providing a threaded seat 39 in the fastening root which engages with a fastening screw 38, attaching itself to the support 36. The support 36 may be provided with through holes 46 to allow the fastening screw 38 to screw into the threaded seat 39 of the fastening root 45 of the perforating element 33. The fastening screw 38 may be inserted from the back 47 of the support 36, in other words from the face of the support facing opposite to the perforating elements 33. The through holes 46 of the support 36 may each comprise an abutment projection 48, for example an internal abutment crown 48, and the fastening root 45 abuts against the abutment crown 48. The fastening root 45 may be provided with an abutment counter-ridge 49, for example a fastening flange 49 which abuts against the abutment crown 48. The abutment projection 48 may form a further abutment surface for the head of the fastening screw 38. The fastening screw 38 may also be screwed to the walls 39′ of the through hole 46 of the support 36.

The perforating elements 33 may be welded or glued to the support 36.

The perforating device 31 may be made of any rigid material, re-sterilizable, and suitable for perforation, such as for example titanium or other surgical metal.

According to an embodiment, the support 36 comprises a thrust surface 37 intended to abut against the plate back 19 of at least one of said first plate 11 or said second plate 12 during the implantation of the implantable device 10, to push the connecting elements 20 inside the through perforations 32 made by the perforating elements 33 inside the body 2 of the tendon 1. In this way it is possible to push at least one of said first plate 11 or said second plate 12 against the surface of the soft tissue 1 to be repaired.

Preferably, the longitudinal extension of each perforating element 33 is greater than the longitudinal extension of the associable first portion 21 of the connecting element 20, but not excessively greater, so as to make its use practical in the operative phase.

The provision, during the implantation of the implantable device 10 of said perforating device 31 equipped with said perforating elements 33, which are fitted onto said connecting elements 20, as well as said connecting elements 20, fitted on said perforating elements 33, allows the insertion of the bio-compatible and bio-resorbable implantable device 10 to be guided through the body 2 of the tendon 1 to be repaired.

The arrangement of the perforating elements 33 on the support 36 of the perforating device 31 is preferably coordinated with and corresponding to the arrangement of the connecting elements 20 on the plates 11, 12. In this way, the perforating elements 33 are also arranged in arrays or rows S1, S2.

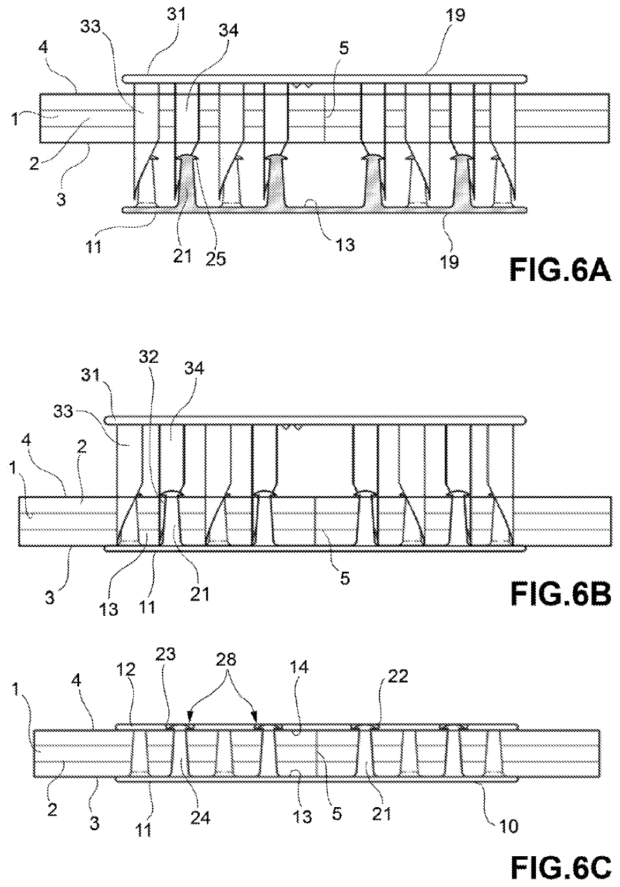

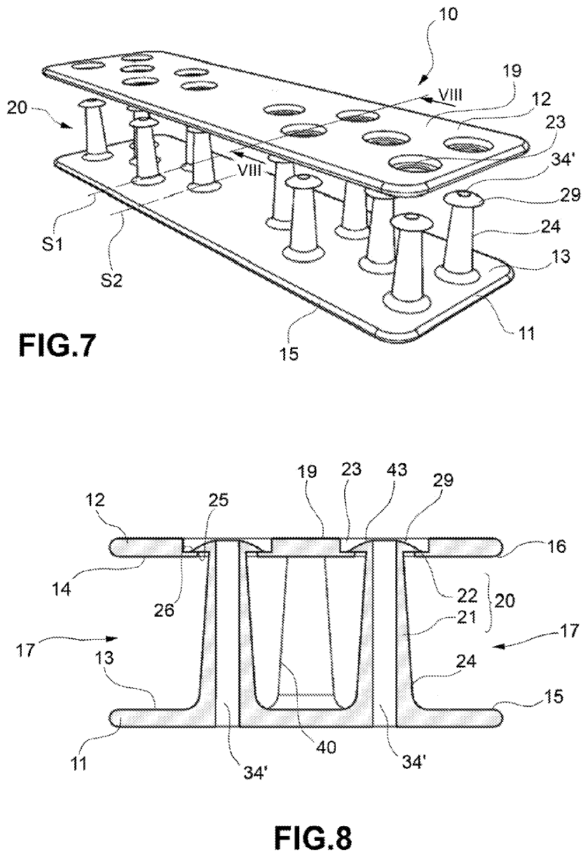

As shown for example in FIG. 6A-6C, the implantation of the implantable device 10 may occur by:
approaching the sharp perforating tips 35 of the perforating elements 33 of the perforating device 31 to one side 4 of the tendon 1 to be repaired near the lesion 5;
making through perforations 32 inside the body 2 of the tendon 1 by inserting said perforating elements 33 inside the body 2 of the tendon 1;
approaching from the side 3 of the body 2 of the tendon 1 the first plate 11 provided with the first portions 21 of the connecting elements 20;
inserting the first portions 21 of the connecting elements 20 into the longitudinal through cavities 34 of the perforating elements 33;
penetrating the first plate 21 into the body 2 of the tendon 1 and at the same time extracting the perforating device 31 from the body 2 of the tendon 1, until the tips of said first portions 21 of the connecting elements 20 emerge from the opposite side 4 of the body 2 of the tendon 1;
applying the second plate 12 on the side 4 of the body 2 of the tendon 1 so that its second portions 22 of the connecting elements 20, for example, holes 23, interlock by latching in an undercut with the first portions 21 of the connecting elements 20, forming said locking portions 28.

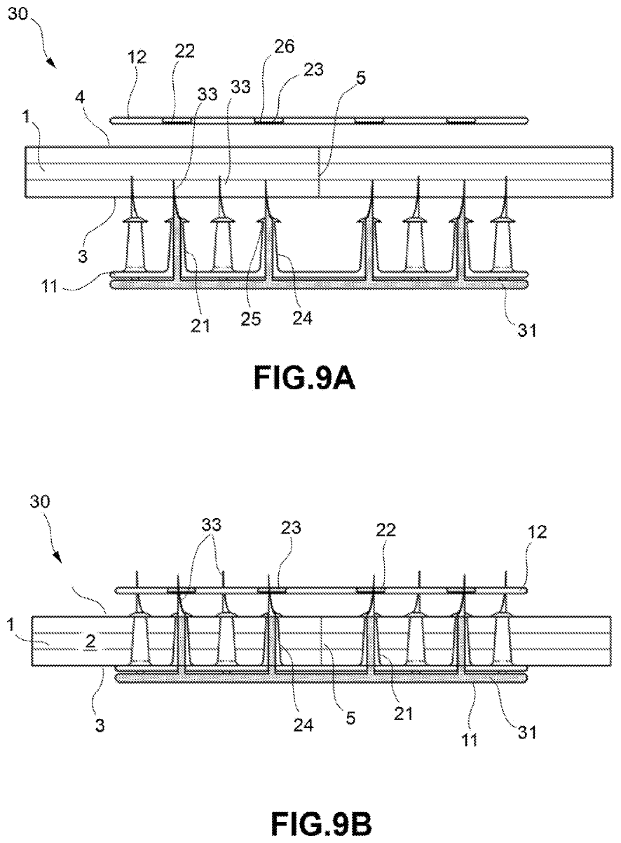

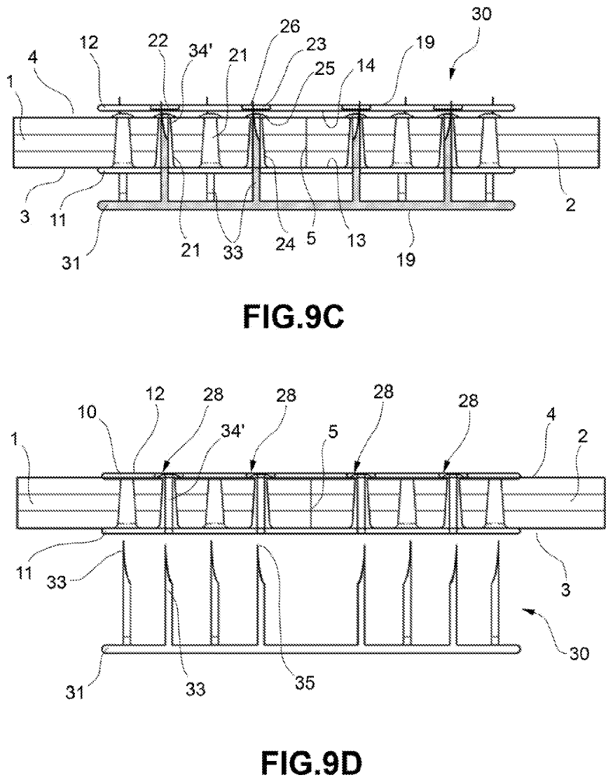

As shown for example in FIG. 9A-9D, the implantation of the implantable device 10 may occur by:
fitting the first portions 21 of the connecting elements 20 of the first plate 11 provided with longitudinal through holes 34′ on the perforating elements 33 of the perforating device 31, so that the sharp perforating tips 35 protrude from the tips of the first portions 21 of the connecting elements 20 of the first plate 11, and preferably bringing the back 19 of the first plate 11 in abutment against said thrust surface 37 of the support 36 of the perforating device 31;
approaching the perforating elements 33 and said first portions 21 of the connecting elements 20 fitted thereon to one side 3 of the tendon 1;
making through perforations 32 inside the body 2 of the tendon 1 by inserting said perforating elements 33 inside the body 2 of the tendon 1 and at the same time guiding the insertion of said first portions 21 of the connecting elements 20 thereon fitted inside the body 2 of the tendon 1, until the tips of said first portions 21 of the connecting elements 20 emerge from the opposite side 4 of the body 2 of the tendon 1;
approaching the second plate 22 to the side 4 of the body 2 of the tendon 1;
extracting the perforating elements 33 of the perforating device 31 and at the same time applying the second plate 12 on the side 4 of the body 2 of the tendon 1 so that its second portions 22 of the connecting elements 20, for example through holes 23, interlock and latch by means of an undercut with the first portions 21 of the connecting elements 20, forming said locking portions 28.

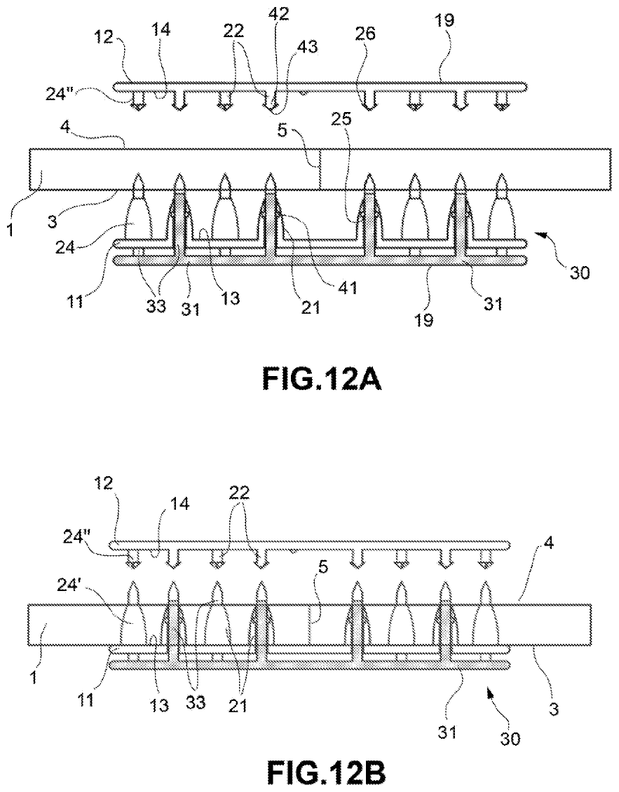

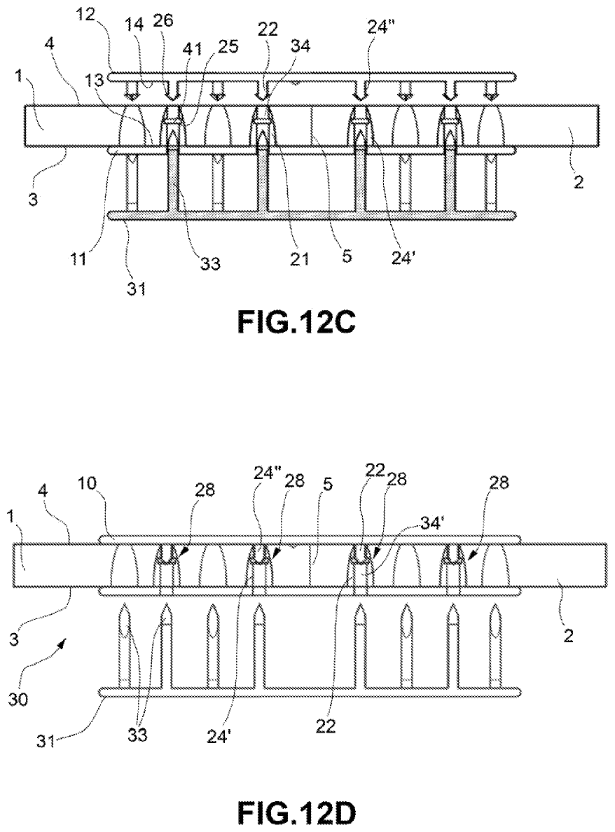

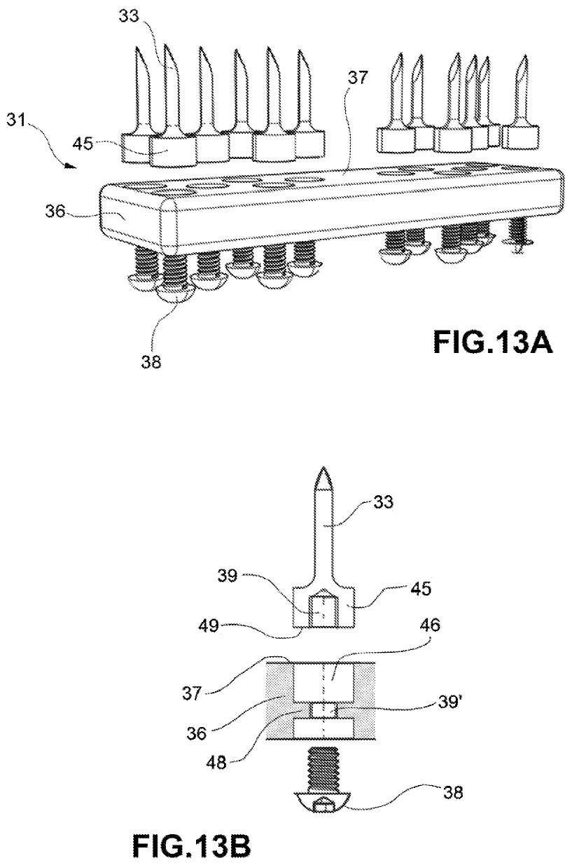

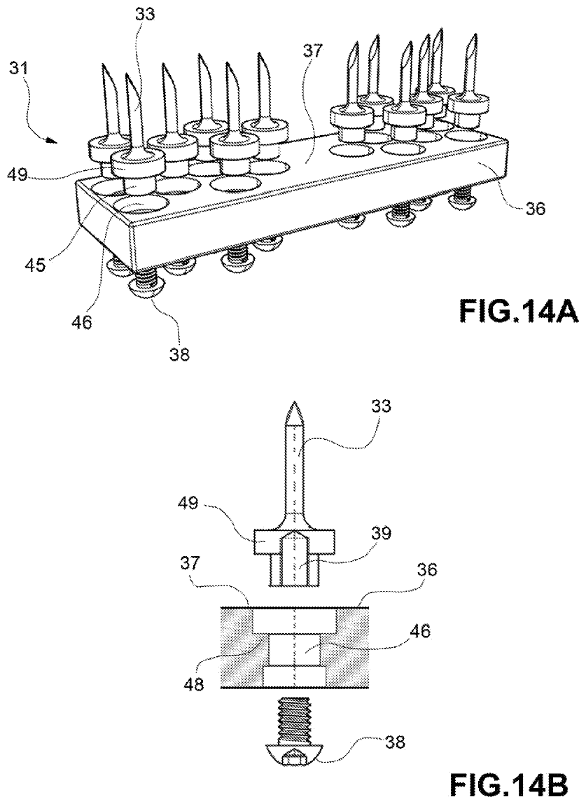

As shown for example in FIG. 12A-12D, the implantation of the implantable device 10 may occur by:
fitting the first portions 21 of the connecting elements 20 of the first plate 11 provided with longitudinal through holes 34′ on the perforating elements 33 of the perforating device 31, so that the sharp perforating tips 35 protrude from the tips of the first portions 21 of the connecting elements 20 of the first plate 11, and preferably bringing the back 19 of the first plate 11 into abutment against said thrust surface 37 of the support 36 of the perforating device 31;
approaching the perforating elements 33 and said first portions 21 of the connecting elements 20 fitted thereon to one side 3 of the tendon 1;
making through perforations 32 inside the body 2 of the tendon 1 by inserting said perforating elements 33 inside the body 2 of the tendon 1 and at the same time guiding the insertion of said first portions 21 of the connecting elements 20 fitted thereon inside the body 2 of the tendon 1, until the tips of said first portions 21 of the connecting elements 20 emerge from the opposite side 4 of the body 2 of the tendon 1;
approaching the second plate 22 to the side 4 of the body 2 of the tendon 1;
extracting the perforating elements 33 of the perforating device 31 and at the same time applying the second plate 12 on the side 4 of the body 2 of the tendon 1 so that its second portions 22 of the connecting elements 20, for example protrusions 24″, interlock by latching in an undercut with the first portions 21 of the connecting elements 20, forming said locking portions 28.

The bio-compatible and bio-resorbable implantable device 10 may be made of a material which is obtained by mixing two or more biopolymers in order to provide optimal mechanical characteristics of tensile strength ensuring their biocompatibility and bioresorbability. For example, said bio-compatible and bio-resorbable implantable device 10 is made by means of a mixture of polylactic acid, PLA, and polycaprolactone, PCL. By acting on the composition of the mixture it is possible to obtain a regulation of the mechanical properties as well as of the degradation rate, in other words the bio-resorption rate, which must allow the connecting elements 20, particularly the protrusions 24, 24′, 24″, and preferably also the plates 11, 12, a bio-resorption time congruent with the time of repair of the soft tissue 1, for example a tendon 1.

Preferably, the bio-compatible and bio-resorbable polymeric mixture degrades through a process of hydrolysis in a physiological environment, the loss of mass may preferably occur through bioerosion in the entire volume (i.e. “in bulk”) or superficially. The connecting elements 20, and particularly the protrusions 24, 24′, 24″, may have different mechanical properties, bio-resorption properties, bio-erosion properties and different composition with respect to the plates 11, 12.

According to an embodiment, connecting elements 20, and particularly the protrusions 24, 24′, 24″, are made of a material having an elastic modulus between 0.2 gigapascals and 4 gigapascals. Preferably, the elastic modulus is comprised between 0.2 gigapascals and 3 gigapascals. According to one embodiment, the elastic modulus is between 0.9 gigapascals and 2.6 gigapascals. The plates 11, 12 may have an elastic modulus equal to or less than that of the connecting elements 20, and particularly of the elastic modulus of the protrusions 24, 24′, 24″. The elastic modulus of the implantable device 10 may be comparable to that of the soft tissue 1.

The bio-compatible and bio-resorbable implantable device 10 may be fabricated by additive manufacturing, such as 3D printing.

The bio-compatible and bio-resorbable implantable device 10 may be fabricated by soft-lithography, soft-tooling or other similar technologies.

By virtue of the features described above provided severally or jointly with each other in particular embodiments, it is possible to obtain a bio-compatible and bio-resorbable implantable device as well as an assembly, which at the same time satisfies the above-described requirements, conflicting with each other, and the aforementioned desired advantages, and in particular:
the bio-compatible and bio-resorbable implantable device may be firmly positioned without needing to be fastened or tied to the tendon to be repaired;
the risk of inflammation of the tendon to be repaired is avoided or at least minimized;
a solution is provided that may be applied to various types of soft tissues, for example tendons and ligaments;
the bioresorbability of the implantable device allows a gradual progressive distribution over time of the physiological tensile loads on the tendon to be repaired during the healing of the tendon, promoting its complete functional recovery;
the need for sutures is avoided;
the plates are interlockable because the connecting elements are interlockable;
it is unnecessary to provide cables or other elements to connect the two plates or keep them connected after the installation;
the pins or protrusions that form the connecting elements do not injure the soft tissue to be repaired but allow a firm locking in a single respective position of the two plates;
the connecting elements between the plates are suitable for inserting between the fibers of the tendon to be repaired, avoiding interrupting or damaging them, thus promoting their healing;
the provision of a perforating device allows jamming stresses on the connecting elements to be avoided, which therefore may include fine mechanical processing, for example with undercutting and/or grooves and bottlenecks, and may be made with mechanical properties, for example stiffness, comparable to those of the soft tissue to be repaired;
the assembly is made up of three plates 11, 12, 31, of which two plates 11, 12 are implantable, bio-compatible and bio-resorbable, interlockable, without requiring sutures or the like, and a third non-implantable perforating plate 31.

A person skilled in the art, in order to satisfy contingent and specific needs, may make numerous modifications and adaptations to the embodiments described above, and replace elements with other functionally equivalent ones, without however departing from the scope of the following claims.

### LIST OF REFERENCES

1 Soft tissue, or tendon, or ligament
2 Body of the tendon
3 First side of the tendon
4 Second side of the tendon
5 Tendon lesion
10 Bio-compatible and bio-resorbable implantable device
11 First bio-compatible and bio-resorbable plate
12 Second bio-compatible and bio-resorbable plate
13 First surface of the first plate
14 Second surface of the second plate
15 Edge of the first plate
16 Edge of the second plate
17 Window
19 Plate back
20 Bio-resorbable and bio-compatible connecting elements
21 First portion, or first piece, of the connecting element
22 Second portion, or second piece, of the connecting element
23 Connecting element hole
24, 24′, 24″ Protrusion of connecting element
25 First abutment surface of the first portion of the connecting element
26 Second abutment surface of the second portion of the connecting element
28 Locking portion, or interlocking portion
29 Head of connecting element
30 Assembly
31 Perforating device of the assembly, or third perforating plate
32 Perforation in the soft tissue
33 Perforating element of the perforating device
34 Longitudinal through channel of the perforating device
34′ Longitudinal through channel of the connecting element
35 Sharp perforating tip
36 Support of the perforating device
37 Thrust surface of the perforating device
38 Fastening screw
39, 39′ Threaded seat
40 Distance
41 Internal seat cavity of connecting element
42 Polygonal base of connecting element
43 Tip of connecting element portion
44 Lateral surface
45 Fastening root of the perforating element
46 Through hole of the support
47 Back of the support
48 Abutment ridge, for example internal abutment crown
49 Abutment counter-ridge, for example abutment flange
S1 First array or first row
S2 Second array or second row

What is claimed is:

1. A bio-compatible and bio-resorbable implantable device for repairing a soft tissue, comprising:
at least two bio-compatible and bio-resorbable plates comprising a first plate and a second plate made as separate and interlockable pieces; and
a plurality of bio-compatible and bio-resorbable connecting elements suitable for connecting said first plate and said second plate;
wherein:
said first plate comprises a first surface suitable for being laid onto a first side of the soft tissue;
said second plate comprises a second surface suitable for being laid onto a second side of the soft tissue;
each connecting element of said plurality of bio-compatible and bio-resorbable connecting elements comprises a first portion integral with the first plate and a second portion integral with the second plate; and
said plurality of bio-compatible and bio-resorbable connecting elements extend from at least one of said first surface of the first plate and said second surface of the second plate for reaching the other one of said first surface of the first plate and said second surface of the second plate for locking in a definable respective position said first plate and said second plate, thereby acting as positioning elements of the first and second plates in respect to the soft tissue.

2. The bio-compatible and bio-resorbable implantable device of claim 1, wherein said plurality of bio-compatible and bio-resorbable connecting elements have same shape and size.

3. The bio-compatible and bio-resorbable implantable device of claim 1, wherein said plurality of bio-compatible and bio-resorbable connecting elements extend from said first surface of the first plate to said second surface of the second plate in a straight line.

4. The bio-compatible and bio-resorbable implantable device of claim 1, wherein said first portion and said second portion of said plurality of bio-compatible and bio-resorbable connecting elements are interlockable according to any of the following modes:
undercut coupling,
latching,
snap-fitting,
force fitting,
hook-loop coupling, and
any combination thereof.

5. The bio-compatible and bio-resorbable implantable device of claim 1, wherein one of said first portion and second portion of each connecting element comprises a hole, either blind or passing through, and the other one of said first portion and second portion of each connecting element comprises a protrusion.

6. The bio-compatible and bio-resorbable implantable device of claim 1, wherein at least one of said first portion and said second portion of each connecting element comprises a protrusion comprising a head with a convex tip.

7. The bio-compatible and bio-resorbable implantable device of claim 1, wherein at least one of said first portion and said second portion of each connecting element comprises a protrusion comprising a convex base, and a lateral surface devoid of sharp edges, for example cylindrical or frusto-conical.

8. The bio-compatible and bio-resorbable implantable device of claim 1, wherein said plurality of bio-compatible and bio-resorbable connecting elements are arranged in arrays, wherein the connecting elements of a first array are offset with respect to the connecting elements of a second array adjacent to said first array.

9. The bio-compatible and bio-resorbable implantable device of claim 1, wherein both said first plate and said second plate delimit at least partially one or more windows suitable for exposing a portion of the soft tissue to surrounding environment.

10. The bio-compatible and bio-resorbable implantable device of claim 1, wherein the connecting elements are made of a bio-compatible and bio-resorbable material having elastic modulus comprised between 0.2 gigapascals and 4 gigapascals.

11. An assembly for repairing a soft tissue, comprising:
at least one bio-compatible and bio-resorbable implantable device for repairing a soft tissue, comprising:
at least two bio-compatible and bio-resorbable plates comprising a first plate and a second plate made as separate and interlockable pieces; and
a plurality of bio-compatible and bio-resorbable connecting elements suitable for connecting said first plate and said second plate;
wherein:
said first plate comprises a first surface suitable for being laid onto a first side of the soft tissue;
said second plate comprises a second surface suitable for being laid onto a second side of the soft tissue;
each connecting element of said plurality of bio-compatible and bio-resorbable connecting elements comprises a first portion integral with the first plate and a second portion integral with the second plate; and
said plurality of bio-compatible and bio-resorbable connecting elements extend from at least one of said first surface of the first plate and said second surface of the second plate for reaching the other one of said first surface of the first plate and said second surface of the second plate for locking in a definable respective position said first plate and said second plate, thereby acting as positioning elements of the first and second plates in respect to the soft tissue; and
a non-implantable perforating device made in separate piece with respect to said bio-compatible and bio-resorbable implantable device;
wherein said non-implantable perforating device comprises a plurality of perforating elements suitable for making perforations in said soft tissue for making positioning paths for said plurality of bio-compatible and bio-resorbable connecting elements.

12. The assembly of claim 11, wherein each of said plurality of perforating elements delimits a longitudinal through channel to allow insertion of said plurality of bio-compatible and bio-resorbable connecting elements, whereby the perforating elements are fit onto the first portion or onto the second portion of the connecting elements during implantation of said first plate or said second plate, respectively.

13. The assembly of claim 11, wherein said non-implantable perforating device comprises a support from which said perforating elements extend.

14. The assembly of claim 13, wherein said perforating elements are made as separate pieces with respect to said support and fastened thereto by threaded fastening means, whereby the perforating elements are screwed to the support.

15. The assembly of claim 13, wherein said support comprises a thrust surface configured to abut against a back plate of at least one of said first plate and said second plate during implantation of the bio-compatible and bio-resorbable implantable device, for pushing the connecting elements within the perforations made by the perforating elements within the soft tissue.

16. The bio-compatible and bio-resorbable implantable device of claim 1, wherein said soft tissue is a tendon.

17. The bio-compatible and bio-resorbable implantable device of claim 1, wherein said plurality of bio-compatible and bio-resorbable connecting elements have a same material composition.

18. The bio-compatible and bio-resorbable implantable device of claim 3, wherein said plurality of bio-compatible and bio-resorbable connecting elements extending from the first surface of the first plate to the second surface of the second plate are all parallel.

19. The bio-compatible and bio-resorbable implantable device of claim 4, wherein interlocking of the first and second portions is made by undercut coupling, and wherein the first portion comprises a first abutment surface facing the first surface of the first plate, so as to couple against a second abutment surface of the second plate, which is opposite or contraposed with respect to the second surface of the second plate.

20. The bio-compatible and bio-resorbable implantable device of claim 1, wherein the first portion of each connecting element comprises a first protrusion extending from said first plate and the second portion of each connecting element comprises a second protrusion extending from said second plate.

21. The bio-compatible and bio-resorbable implantable device of claim 10, wherein said bio-compatible and bio-resorbable material is a blend of two or more biopolymers.

22. The bio-compatible and bio-resorbable implantable device of claim 21, wherein said blend is a blend of polylactic acid and polycaprolactone.

23. The assembly of claim 11, wherein the connecting elements each delimit a longitudinal through channel for insertion of said plurality of perforating elements of the perforating device, so that the connecting elements are fit onto the perforating elements during implantation of at least one of said first plate and second plate.
